(19) United States
(12) Plant Patent Application Publication
(10) Pub. No.: US 2006/0218686 P1
(43) Pub. Date: Sep. 28, 2006

(54) PELARGONIUM PLANT NAMED 'AMRI DERED'

(76) Inventor: Mitchell E. Hanes, Morgan Hill, CA (US)

Correspondence Address: JONDLE & ASSOCIATES P.C., 858 HAPPY CANYON ROAD SUITE 230, CASTLE ROCK, CO, 80108, US

(21) Appl. No.: 11/091,203

(22) Filed: Mar. 28, 2005

(51) Int. Cl. A01H 5/00

(52) U.S. Cl. PLT/330

(57) ABSTRACT

A Pelargonium cultivar particularly distinguished by its bright, rich red flower color and double flowers is disclosed.

### GENUS AND SPECIES

Pelargonium×hortorum L. H. Bailey

### VARIETY DENOMINATION

‘Amri Dered’

### BACKGROUND OF THE NEW PLANT

The present invention comprises a new and distinct cultivar of Pelargonium, botanically known as Pelargonium×hortorum L. H. Bailey, and hereinafter referred to by the cultivar name ‘Amri Dered’. The new cultivar was propagated from a seedling resulting from the cross of 8691-2, a dark red-flowered, unnamed, unpatented proprietary Pelargonium line and ‘Rumba Fire’, a scarlet-flowered Pelargonium (U.S. Plant Pat. No. 12,489).

The new cultivar was created in 2000 in Gilroy, Calif. and has been asexually reproduced repeatedly by vegetative cuttings in Gilroy, Calif. over a four year period. It has also been trialed at Gilroy, Calif., Litchfield, Mich., and Andijk, The Netherlands. It has been found to retain its distinctive characteristics through successive asexual propagations.

### DESCRIPTION OF PHOTOGRAPH

This new geranium plant is illustrated by the accompanying photograph which shows blooms, buds, and foliage of the plant; the colors shown are as true as can be reasonably obtained by conventional photographic procedures. The photograph is of a 4 month old plant grown in a lexan-covered greenhouse with normal zonal pelargonium culture.

FIG. 1. shows overall plant habit while the inset photograph show a closer view of the mature inflorescence.

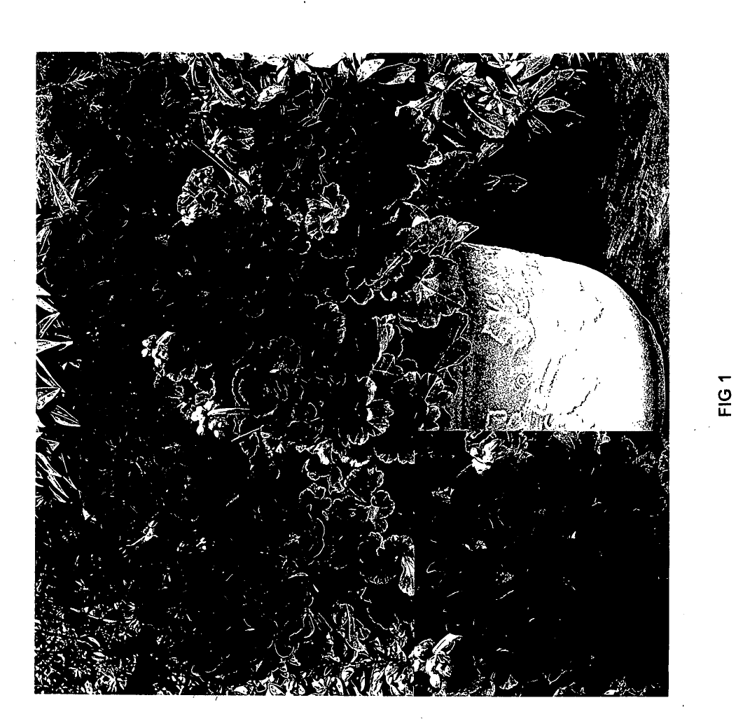

### DESCRIPTION OF THE NEW CULTIVAR

The following detailed descriptions set forth the distinctive characteristics of ‘Amri Dered’. The data which defines these characteristics were collected from asexual reproductions carried out in Gilroy, Calif. The plant history was taken on seven-month-old plants grown in 1 gallon pots in a poly-covered greenhouse during the fall/winter season. The color readings were taken under natural light in the greenhouse in the late winter season. Color references are primarily to the RHS Colour Chart of The Royal Horticultural Society of London (RHS) (2001). Texture description data were collected by viewing plant parts with a dissecting microscope.

### DETAILED BOTANICAL DESCRIPTION

Classification:
   Botanical.—Pelargonium×hortorum L. H. Bailey.
   Common Name.—Geranium.
Growth:
   Form.—Upright, outwardly spreading and rounded growth habit.
   Growth and branching habit.—Good vigorous habit, well branched.
   Height.—15-23 cm as measured from soil level to top of plant, excluding blooms; 20-25 cm from soil level to top of umbels.
   Width.—35-45 cm excluding umbels.
   Time to produce a finished flowering plant.—8-10 weeks.
   Outdoor plant performance.—Has good weather tolerance; used in hanging baskets, patio or mixed containers.
   Time to initiate and develop roots.—12-28 days.
   Root description.—White, slightly pubescent, fibrous yet coarse.
Leaves:
   Arrangement.—Alternate, simple.
   Immature leaf.—Color: Upper surface: RHS 146A Lower surface: RHS 146B Length: 4.5-4.7 cm Width: 5.5-7.5 cm.
   Mature leaf: Color: Upper surface: Between RHS 146A and RHS 146B Lower surface: RHS 146B Length: 6.0-6.5 cm Width: 10.0-10.5 cm.
   Zonation color.—Faint shading of RHS N199B.
   Zonation diameter.—1.5-2.0 cm.
   Apex description.—Rounded.
   Base description.—Cordate.
   Shape.—Reniform.
   Margin.—Crenate; somewhat sinuate.
   Texture.—Pilose, velvety.
   Venation.—Palmate.
   Venation color.—RHS 144A.
   Petioles.—Length: 9.0-10.0 cm Diameter: 0.35 cm Color: RHS 144A Texture: Pilose; some glandular hairs.
Stems:
   Length.—20-25 cm.
   Internode length.—1.0-1.5 cm.
   Color.—RHS 144B.
   Texture.—Pilose; many glandular hairs.
Flower buds:
   Diameter.—0.7-0.9 cm.
   Length.—0.9-1.3 cm.
   Shape.—Elliptical.
   Color when sepals first divide.—RHS 45B.
Inflorescence:
   Type.—An umbel composed of 13-16 flowers.
   Umbel diameter.—8.0-9.0 cm.
   Umbel depth (height).—5.6 cm.
   Blooming habit.—Continuous blooming throughout the growing season; good floriferousness.
   Peduncle.—Length: 14-15 cm Diameter: 0.5 cm Texture: Pilose; many glandular hairs Color: RHS 144A.
   Pedicel.—Length: 1.7-1.8 cm Diameter: 0.1 cm Texture: Many glandular hairs Color: RHS 144A.
Flowers:
   Flower form.—Polypetalous; mostly imbricate petals and petaloids, semi-double form consisting of 7-9 petals and 1-3 petaloids.
   Flower diameter.—4.5-5.3 cm.
   Flower persistence.—An open flower will hold its petals until the flower begins to dehisce, 7-10 days.
   Fragrance.—None.
Petals/petaloids:
   Petal size.—Length: 2.5-2.9 cm Width: 2.3-2.7 cm.
   Shape.—Obovate.
   Apex.—Rounded.
   Base.—Acute.
   Margin.—Entire.
   Texture.—Papillose.
   Upper petal color.—Upper surface: Velvety RHS 44B but brighter and richer, with basal veining a faint RHS 46B Lower surface: RHS 44B with streaks of RHS 44A basally.
   Lower petal color.—Upper surface: Velvety RHS 44B but brighter and richer, with basal veining a faint RHS 46B Lower surface: RHS 44B.
   Upper petaloid color.—Upper surface: Velvety RHS 44B but brighter and richer, with basal veining a faint RHS 46B.
   Lower petaloid color.—Upper surface: Velvety RHS 44B but brighter and richer, with basal veining a faint RHS 46B.
   Color change with age.—Colors do not change with age but may change with temperature and time of year.
Sepals:
   Number of sepals.—5, one larger than the others; fused at base.
   Length of sepals.—0.9-1.0 cm.
   Width of sepals.—0.2-0.4 cm.
   Shape.—Oblanceolate.
   Apex.—Acuminate.
   Texture.—Pilose, villous; many glandular hairs.
   Color.—RHS 144A.
Reproductive organs:
   Stamens.—4-6, sometimes fused to petaloids.
   Filament color.—RHS N155B but whiter.
   Filament length.—0.6 cm.
   Pollen color.—RHS N30A.
   Pollen amount.—Scant to moderate.
   Pistil.—1.
   Pistil length.—0.9 cm.
   Stigma color.—RHS 42A.
   Style color.—RHS 39A.
   Style length.—0.8 cm.
   Fruit set.—Not observed.
Disease and insect resistance: Not observed.

### COMPARISON WITH KNOWN CULTIVARS

When the instant plant is compared to the female parental cultivar 8691-2, ‘Amri Dered’ has double flowers, no zone on the leaf, and upright habit with better branching than 8691-2. When ‘Amri Dered’ is compared to the male parental cultivar ‘Rumba Fire’ (U.S. Plant Pat. No. 12,489), ‘Amri Dered’ has dark red flowers, is more free flowering and has a lighter and narrower zone on the leaves than ‘Rumba Fire’.

‘Amri Dered’ differs from the comparison variety, ‘Rocky Mountain Red 2003’ (U.S. Plant Pat. No. 13,961) by having larger leaves, a shade darker flower and more petals than ‘Rocky Mountain Red 2003’.

I claim:

1. A new and distinct cultivar of Pelargonium plant as shown and described herein.
